(19) United States
(12) Patent Application Publication
(10) Pub. No.: US 2007/0149922 A1
(43) Pub. Date: Jun. 28, 2007

(54) Combined fixed volume retention cuff and relief valve

(75) Inventors: James G. Schneider, Chesterfield, MO (US); Nick Martino, Fernandina Beach, FL (US); Raymond O. Bodicky, Oakville, MO (US)

Correspondence Address: MARSHALL, GERSTEIN & BORUN LLP, 233 S. WACKER DRIVE, SUITE 6300, SEARS TOWER, CHICAGO, IL, 60606, US

(73) Assignee: Bowel Management Systems, LLC, Fernandina Beach, FL (US)

(21) Appl. No.: 11/319,309

(22) Filed: Dec. 28, 2005

(51) Int. Cl. A61M 29/00; A62B 9/02

(52) U.S. Cl. 604/99.01; 128/205.24

(57) ABSTRACT

The invention is a system (10, 110) for regulating volume in a retention cuff/balloon (20) in an indwelling catheter; i.e., the combination of fixed-volume inflatable retention cuff mountable on a patient proximal end of an indwelling catheter (14) and relief valve (23, 123) connected to the cuff. The relief valve has ends for selective introduction of fluid to the cuff and for connection to an inflation tube. A valve port (24, 124) permits escape of excess fluid when a maximum cuff volume has been exceeded, and inflation tube (22) connects cuff (20) to the relief valve. The inflation tube has ends in fluid-tight connection to an interior of the cuff, and the relief valve to thereby permit the cuff and valve to be in fluid communication with each other for selective filling of the cuff to insert and safely retain an indwelling catheter in a patient for an extended period.

### BACKGROUND OF THE INVENTION

1. Field of the Invention

The present invention relates, generally, to catheter retention devices, and, more particularly, to a retention cuff of the type used on bowel maintenance catheters, having a fixed maximum volume and being combined with a fluid relief valve.

2. Related Art

The present invention is considered for use primarily (but not necessarily exclusively) with or as an improvement to known bowel management devices, such as, for example, those presently available and marketed by Zassi Medical Evolutions, Inc and Bowel Management Systems, LLC. These systems are described in U.S. Pat. No. 5,569,216 and pending U.S. application Ser. No. 10/225,820, published as US 2004/0039348 on Feb. 26, 2004, the entire disclosures of which patent and application are incorporated herein by reference. The present invention can also be incorporated into other catheter devices having balloon retention mechanisms. For convenience and simplicity of the disclosure, the invention is described specifically in terms of bowel management systems, herein, although other uses in mammals and especially people can be readily conceived.

There is a continuing need in the art to maintain a selected operative position of a bowel maintenance device, and to do so without trauma to the patient. A variety of catheters used in the body have balloons or cuffs to hold them in place and to create a seal between the organ they are placed in and the outside environment (e.g., Foley catheters, endotracheal tubes). In the majority of these catheters there is no safeguard to prevent the balloon or cuff from being over inflated by the caregiver, the end result of which is an oversized balloon or cuff that can cause trauma to the organ in which it resides. By contrast, angioplasty catheter balloons are fixed-size balloons. They are sized to dilate to specific diameters to open clogged arteries. Their size is limited by the material characteristics of the balloon. Any attempts to over-inflate the balloon result in very little increase in balloon size. Various materials used to fabricate angioplasty balloons include: PET, Nylon, Polyurethane, Polyethylene and PVC.

There also exist pressure limiters available for use with endotracheal tubes. These devices are placed in-line with a cuff inflation lumen and limit the pressure within the endotracheal balloon cuff (e.g., Mallinckrodt's Hi-Lo® Tracheal Tube with Lanz® Pressure Regulating Valve).

Known angioplasty balloons are not particularly compliant and are not designed for long-term indwell. If left in place for extended periods of time they create the potential to traumatize the vessel via vessel wall erosion. Hence the size limiting properties of angioplasty balloons are not a useful approach to controlling bowel management system retention cuffs.

Existing pressure regulating valves for endotracheal tubes are too sensitive for use in the present application. Their function is to limit the pressure the cuff is exerting on the trachea wall. This would be undesirable in bowel management devices, which should ignore (except in extreme cases) transient environmental pressure variances (which can be the result of normal physiologic occurrences—e.g. peristalsis) and maintain a specific cuff inflation volume. Generally, known catheters having inflatable cuffs are those in which the cuff is formed of elastic material and there is no mechanism by which to limit the volume of inflation media infused in the cuff. Thus, these known cuffs can readily be over-inflated and are not optimal for long-term indwelling use.

Accordingly there is a need in the art for an indwelling bowel management device with a fixed-volume retention cuff of the balloon type and having a relief valve. There further remains a continuing need for economy, durability and reliability of such devices.

### SUMMARY OF THE INVENTION

There are two key components of the present bowel management system: (1) a fixed maximum volume retention cuff/balloon and (2) a relief valve in the retention cuff/balloon inflation fluid path of the system. This combination, as described below, eliminates the ability of the end user to purposefully (e.g., in an attempt to prevent leakage) or accidentally over-inflate the balloon/cuff of an indwelling catheter.

The fixed volume inflatable cuff has the following properties. It is low pressure, and therefore, compliant with the rectal contours at its normal inflation volume. However, the size of the balloon is substantially fixed because the material of which it is formed is quite inelastic as compared to other inflation cuffs, such as those used on endotracheal tubes. If a user were to try and over inflate the cuff of the present system, the amount of pressure to do so would be extreme as compared to the normal pressure required to inflate the cuff.

The balloon or “cuff” may be molded of silicone or polyurethane with nylon fiber/web reinforcement, silicone or polyurethane with polyester fiber/web reinforcement, Nylon, flexible PVC, Polyethylene or polyethyltetraethylene (PET). These materials can be molded in a pre-distended state and because they have very little elongation they will inflate only to a substantially fixed, predetermined size. Any attempt to put more (excess) volume into the cuff will dramatically increase the amount of pressure the user will be required to exert to infuse the additional media.

A variety of pressure relief valves exist which may be suitable for this general type of application. And wherein, valves for detecting very small changes in pressure are difficult to design and expensive to manufacture, the steep slope of the balloon/cuff volume vs. pressure curve near the over-inflation point greatly simplifies the pressure bleed valve needed for this application. Accordingly, the new relief valve need not be expensive to make. The ratio of the balloon/cuff volume vs. pressure curve allows the valve to be constructed to trigger at pressures higher than what normally would be generated as a result of environmental variances, (e.g. peristalsis). By combining a fixed-volume balloon/cuff as described above with the addition of a simple relief valve the ability of the end user to purposely or accidentally over inflate the balloon/cuff is virtually eliminated.

Thus, it is among the advantages of the present invention to provide a combined fixed volume retention cuff and relief valve in the same system to facilely and economically permit long-term bowel management by care givers with a low level of training, yet with reliable results in terms of secure and safe inflation of a catheter retention cuff.

Accordingly, in furtherance of the above goals and advantages, the present invention is, briefly, a system for regulating volume in a retention cuff/balloon in an indwelling catheter. The system is the combination of a fixed-volume inflatable retention cuff mountable on a patient proximal end of an indwelling catheter and a relief valve connected to the fixed-volume inflatable retention cuff, the relief valve having a first end for selective introduction there through of inflation fluid via the relief valve and an inflation tube to the fixed-volume retention cuff, a second end for connection to the inflation tube and a valve wall extending between the first end and the second end of the valve, and a port to permit escape of excess fluid when a pre-selected maximum retention cuff volume has been exceeded. The system also includes an inflation tube connecting the fixed-volume inflatable retention cuff to the relief valve, the inflation tube having a first end and a second end and extending therebetween. The first end of the inflation tube is in fluid-tight connection to an interior of the fixed-volume retention cuff, and the second end of the inflation tube being in fluid-tight connection with the second end of the relief valve to thereby permit the fixed-volume inflatable retention cuff and the relief valve to be in fluid communication with each other for selective filling of the fixed-volume inflation cuff with fluid when it is desired to insert and safely retain an indwelling catheter in an organ of a patient for an extended period of time.

The invention is also, briefly, a catheter system including a catheter having a lumen end adapted for insertion in a patient organ and a balloon cuff attached to the catheter adjacent the lumen end, the balloon cuff being adapted for insertion in the patient organ. The balloon cuff is inflatable only to a pre-selected volume. The system includes a tube having a first end in fluid communication with an interior of the balloon and a second end attached to a relief valve. The relief valve is in operative communication with the tube such that cuff inflation fluid is released externally to the patient organ when the space within the balloon and tube exceeds a pre-selected trigger pressure.

Further areas of applicability of the present invention will become apparent from the detailed description provided hereinafter. It should be understood that the detailed description and specific examples, while indicating the preferred embodiment of the invention, are intended for purposes of illustration only and are not intended to limit the scope of the invention.

### BRIEF DESCRIPTION OF THE DRAWINGS

The present invention will become more fully understood from the detailed description and the accompanying drawings, wherein:

FIG. 1 is a perspective view of a first embodiment of a bowel management system with the new combined fixed volume retention cuff and relief valve.

FIG. 1A is a longitudinal sectional view of the valve portion of the system of FIG. 1 showing the first and second ends of the valve.

FIG. 2A is a partial longitudinal sectional view of the relief valve of FIG. 1 in a closed position.

FIG. 2B is a partial longitudinal sectional view of the relief valve of FIG. 1 in an open position, permitting fluid release.

FIG. 3 is a perspective view of another embodiment of the combined fixed volume cuff/relief valve of the present invention, partially broken away.

FIG. 4A is a longitudinal sectional view the relief valve of FIG. 3 in a closed position.

FIG. 4B is a longitudinal sectional view of the relief valve of FIG. 3 in an open position.

FIG. 5 is a cross sectional view of a known bowel management system of a type with which the device of the present invention can be used, positioned for normal use, in a patient, in the open flow configuration, so that body wastes can readily flow distally from the patient through the main catheter.

Throughout the drawings, like parts are indicated by like element numbers.

### DETAILED DESCRIPTION OF PRACTICAL EMBODIMENTS

The following description of the preferred embodiment(s) is merely exemplary in nature and is in no way intended to limit the invention, its application, or uses.

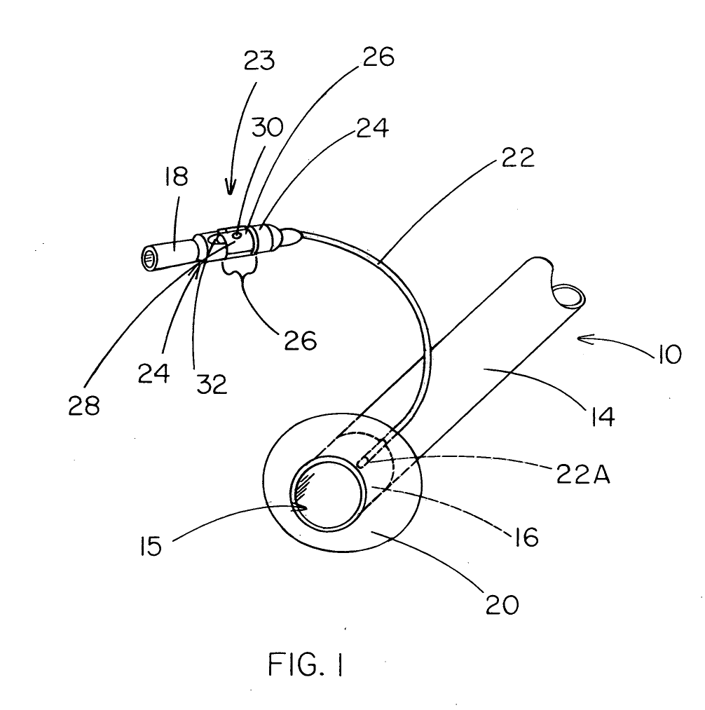

FIGS. 1 and 1A depict a bowel management catheter system 10 having combined therein a fixed-volume retention cuff 20 and fluid volume relief assembly 23, including both fluid relief valve and flow shut-off mechanisms. The overall bowel management system 10 is developed for attachment to a conduit or main catheter 14 having an internal lumen 15 for passage there through of a patient's body waste when catheter 14 is in the open-flow configuration shown in FIG. 5. As depicted in FIG. 5, use of a bowel management system depends upon insertion of the main catheter 14 into through the patient's anus and thereafter the maintenance of catheter 14 in the patient's rectum. Maintenance of the normal operative position of system 10 is achieved by inflation of an internal balloon or cuff 20, which is shown in its inflated position in FIG. 1, mounted on patient proximal end 16 of catheter 14. The present system, however, varies from that shown in FIG. 5 by virtue of the combined fixed-volume inflatable cuff and relief valve described herein.

Cuff/balloon 20 is inflatable via inflation port 22A, which is the site at which balloon 20 is penetrated in fluid-tight manner via one end of a preferably single lumen tube 22. A fluid relief assembly 23 at the opposite and free end of inflation tube 22 provides a mechanism by which to attach a source of inflation fluid and to relieve fluid volume if a predetermined volume is exceeded. The attachment mechanism preferably takes the form of a female luer connector 18 or other suitable structures by which to connect a source of inflation fluid, such as a syringe, for example. Luer connector 18 may be fitted with, for example, Halkey-Roberts one-way (in) valve assembly 19, if desired, as illustrated schematically in FIG. 1A, or other suitable shut-off mechanism.

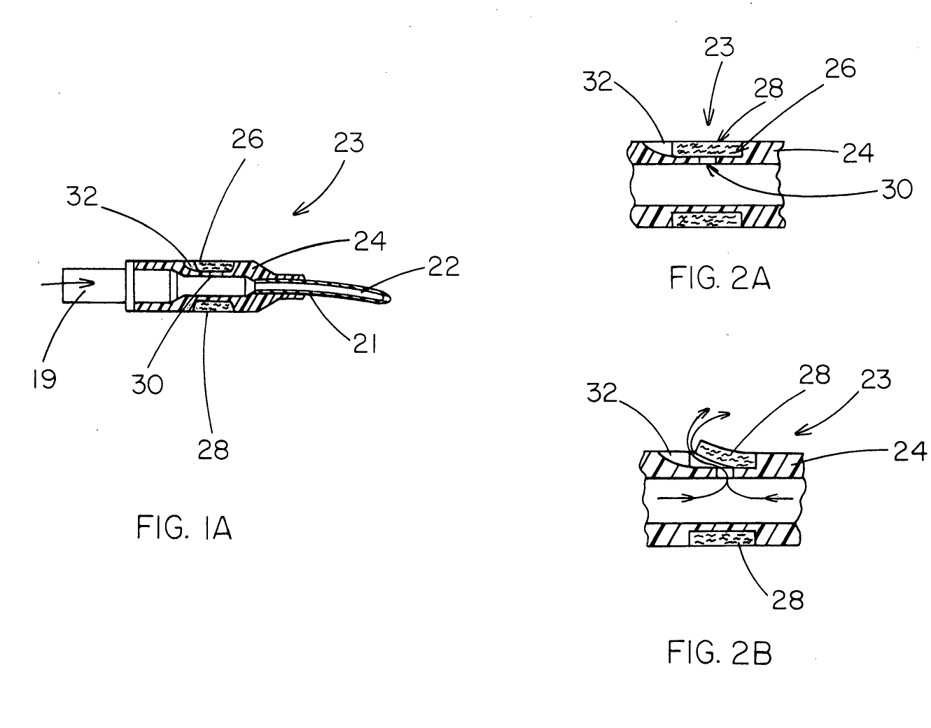

FIGS. 2A and 2AB illustrate that one-way fluid relief assembly (valve) 23 is composed of a flow shut-off, which prevents flow into or out of the relief assembly and in so doing permits the build-up of pressure caused by excess fluid within the relief assembly. A tubular fluid relief port 24, having a central portion, includes an undercut area 26 dimensioned to seat in close cooperation with elastomeric sleeve 28. Sleeve 28 has a predetermined thickness and durometer hardness, and covers a fluid relief through-hole 30. Through-hole 30 communicates between the external atmosphere and the internal lumen of tube 22 (via port 24). Inflation tube 22 is bonded in fluid-tight manner to valve 23, for example as at 21 in FIG. 1A. Thus balloon 20 has fluid communication with the internal lumen of port 24. Sleeve 28 covers through-hole 30 with a fluid-tight seal, provided that the inflation fluid within the lumen of port 24 remains at or below a pre-selected trigger pressure. When the pre-selected cuff fluid volume threshold is exceeded pressure within the relief assembly increases. As the pressure approaches and ultimately exceeds the trigger pressure, an outward force is exerted on the valve elastomeric sleeve 28, to thereby release excess system pressure by adjusting the volume; i.e. by permitting limited fluid escape.

FIG. 2B illustrates the performance of elastomeric sleeve 28 when the volume threshold is exceeded. Elastomeric sleeve 28 extends in response to pressure from excess fluid volume via through-hole 30 and thereby creates a fluid passage for inflation fluid from the internal lumen of port 24 to the outside atmosphere via bleed-off channel 32. Air or other fluid within port 24 and tube 22 exits via through-hole 30 and channel 32 until the pressure from excess fluid volume within port 24 is reduced to or below a pre-selected threshold. At that point, elastomeric sleeve 28 closes, due to its inherent elasticity.

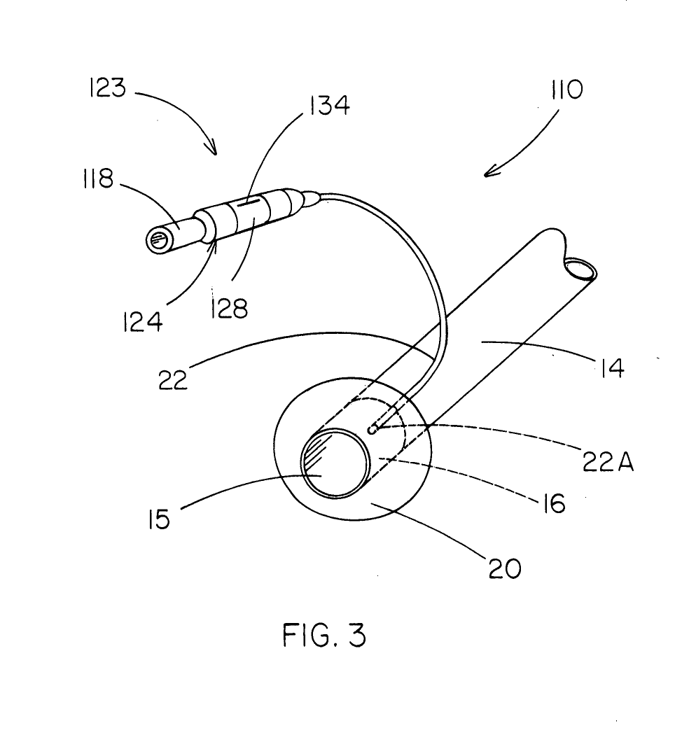

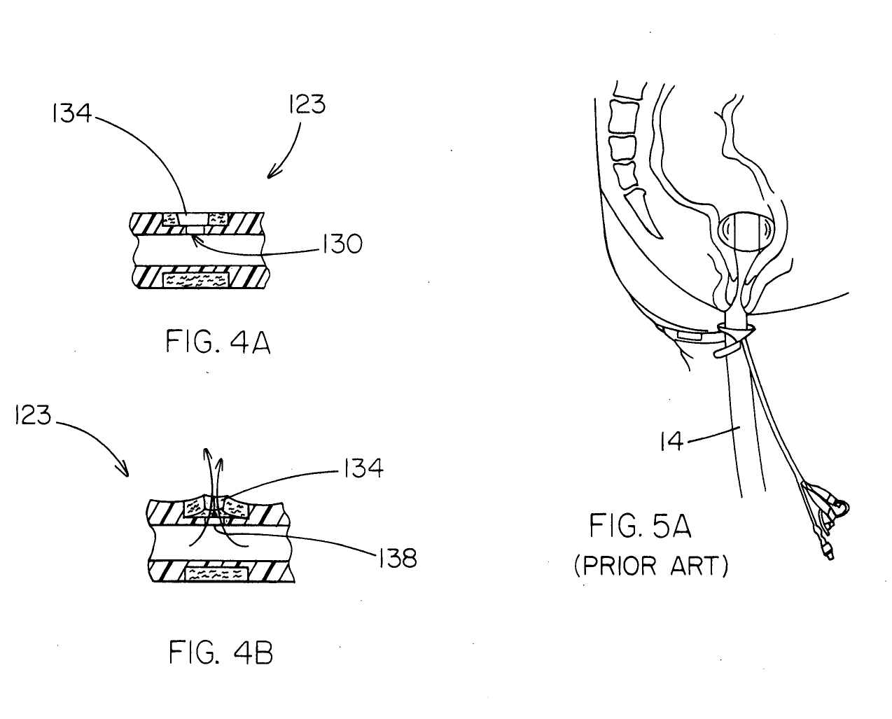

FIG. 3 depicts a second embodiment of the system, generally designated 110, and having the same bowel management system components 14, 20 and 22. System 110 has a relief assembly 123 with a port 124, undercut area 126, an elastomeric sleeve 128 and a through-hole 130: Relief assembly 123 is best seen in FIGS. 4A and 4B. Unlike the first embodiment, in this version, there is no bleed-off channel 32 formed in the port wall. Instead, elastomeric sleeve 128 includes an openable and reclosable slit 134. During inflation of cuff 20, if the pressure from excess fluid within the lumen of port 124 exceeds the pre-selected threshold, slit 134 is forced open to a degree sufficient to allow the escape of air or other inflation fluid from the internal lumen. When the pressure within the internal lumen of port 124 is reduced to within an acceptable range, the inherent elasticity of elastomeric sleeve 128 causes slit 134 to close. This process is repeatable as may be necessary to inflate or deflate cuff 20, as is also true of the embodiment of FIG. 1.

Increasing the fluid volume in cuff 20, as occurs during inflation, results in very little pressure increase in the cuff until the cuff reaches its substantially fixed, predetermined volume, at which point the amount of pressure the user would have to exert to put additional inflation fluid in the cuff would rapidly increase, as would the pressure in the entire retention cuff/balloon inflation fluid path, include the relief port 24, 124. When the pressure within port 24, 124 exceeds a trigger point within a predetermined preferred range the corresponding relief valve 23, 123 will operate one-way, to permit fluid release via associated opening, hole or slit 30, 134. At the point where the relief valve is activated the patient caregiver ceases introduction of inflation fluid. The system can them be left as is, effectively “self-sealed,” or if desired, it can be further sealed, for example, by placing a cap (not shown) over female luer connector 18.

In either disclosed embodiment of the invention, selection of the desired internal pressure may be accomplished by adjusting the elasticity of elastomeric sleeve 28, 128. It is within the scope of the present invention to have interchangeable sleeves of a variety of thickness and/or durometer hardnesses, corresponding to a variety of a selectable threshold pressures.

As various modifications could be made to the exemplary embodiments, as described above with reference to the corresponding illustrations, without departing from the scope of the invention, it is intended that all matter contained in the foregoing description and shown in the accompanying drawings shall be interpreted as illustrative rather than limiting. Thus, the breadth and scope of the present invention should not be limited by any of the above-described exemplary embodiments, but should be defined only in accordance with the following claims appended hereto and their equivalents.

What is claimed is:

1. A system (10, 110) for regulating volume in a retention cuff/balloon in an indwelling catheter;
the system comprising the combination of:
a fixed-volume inflatable retention cuff (20) mountable on a patient proximal end of an indwelling catheter (14);
a relief valve (23, 123) connected to the fixed-volume inflatable retention cuff, the relief valve having a first end for selective introduction there through of inflation fluid via the relief valve and an inflation tube (22) to the fixed-volume retention cuff, a second end for connection to the inflation tube and a valve wall extending between the first end and the second end of the valve, and a port (24, 124) to permit escape of excess fluid when a preselected maximum retention cuff volume has been exceeded; and
an inflation tube (22) connecting the fixed-volume inflatable retention cuff (20) to the relief valve, the inflation tube having a first end and a second end and extending therebetween, the first end of the inflation tube being in fluid-tight connection to an interior of the fixed-volume retention cuff, and the second end of the inflation tube being in fluid-tight connection with the second end of the relief valve to thereby permit the fixed-volume inflatable retention cuff and the relief valve to be in fluid communication with each other for selective filling of the fixed-volume inflation cuff with fluid when it is desired to insert and safely retain an indwelling catheter (14) in an organ of a patient for an extended period of time.

2. The system of claim 1, wherein the fixed-volume inflatable retention cuff (20) is formed of a material which cannot be stretched to enlarge the volume of the cuff; so that filling the cuff with fluid to a volume beyond the predetermined fixed volume thereof will cause the pressure in the cuff to increase to a point that exceeds a pre-selected trigger point range and will result in activation of the relief valve and release of fluid until internal cuff pressure decreases to a point that the valve automatically closes.

3. The system of claim 2, wherein the material of which the fixed-volume inflatable retention cuff (20) is formed is selected from group consisting of nylon, PVC, polyethylene, PET, silicone with nylon fiber reinforcement, silicone with nylon web reinforcement, silicone with polyester fiber reinforcement, silicone with polyester web reinforcement, polyurethane with polyester fiber reinforcement, polyurethane with polyester web reinforcement and polyurethane with nylon reinforcement.

4. The system of claim 1, wherein the cuff (20) is molded into a pre-distended configuration.

5. The system of claim 1, wherein the port of the relief valve (22) comprises:
an undercut area (26) terminating in a bleed-off channel (32) and defining a through-hole (30) from the lumen of the inflation tube (22) to the exterior of they valve; and
a sleeve (28) sized and shaped to seat in the undercut area (26) covering the through-hole, the sleeve being of pre-determined strength and hardness to permit flexing sufficiently to permit escape of excess fluid from the system (10) via the inflation tube, via the through-hole and out the channel to the exterior of the relief valve after the fixed-volume inflatable retention cuff (20) is filled beyond the predetermined maximum volume thereof.

6. The system of claim 1, wherein the port (124) of the relief valve (123) comprises:
an undercut area (126) defining a through-hole (130) from the lumen of the inflation tube (22) to the exterior of the valve;
a sleeve (128) being sized and shaped to seat in the undercut area covering the through-hole, and having a slit (134), the sleeve being of pre-determined strength and hardness to permit flexing sufficiently to permit escape of excess fluid volume from the system (110) via the inflation tube, via the through-hole and out to the exterior of the relief valve via the slit when the fixed-volume inflatable retention cuff (20) is filled beyond the predetermined maximum volume thereof.

7. The system of claim 1, wherein the relief valve (23, 123) is a one-way valve.

8. A catheter system comprising:
a catheter (14) having a lumen end adapted for insertion in a patient organ;
a balloon cuff (20) attached to said catheter adjacent said lumen end, said balloon cuff also being adapted for insertion in the patient organ;
said balloon cuff being inflatable only to a preconfigured volume;
a tube (22) having a first end in fluid communication with an interior of said balloon and a second end attached to a relief valve (23, 123); and
a relief valve (23, 123) in operative communication with said tube such that cuff inflation fluid is released externally to the organ when the space within said balloon and tube exceeds a preconfigured threshold volume.

9. The catheter system of claim 8 wherein said relief valve is further comprised of:
a relief port (24, 124);
a seat (26, 126) in said relief port (24, 124);
a through-hole (30, 130) communicating from the lumen of said tube (22) to the external atmosphere through said seat; and
an elastomeric sleeve (28, 128) dimensioned and configured to fit in close cooperation with said seat and to cover said through-hole, said elastomeric sleeve having a preconfigured elasticity such that when the space within said balloon and catheter exceeds a preconfigured threshold pressure, said elastomeric sleeve deforms to create fluid communication between said space and the external atmosphere through said through-hole (30, 130).

10. The catheter system of claim 9 wherein said relief port includes a channel (32), said channel being configured to direct a release of fluid by permitting bleed-off of excess inflation fluid when the predetermined volume of the retention cuff (20) is exceeded.

11. The catheter system of claim 9 further comprising a slit (134) in said elastomeric sleeve (128), said fluid communication of said space with the external atmosphere being through said slit to permit release of excess fluid from the system when the volume of fluid in the cuff (20) exceeds a predetermined volume.

12. The catheter system of claim 8, wherein the fixed-volume inflatable retention cuff (20) is formed of a material which cannot be stretched to enlarge the volume of the cuff; so that filling the cuff with fluid to a volume beyond the predetermined fixed volume thereof will cause the pressure in the cuff to increase to a point that exceeds a pre-selected trigger point range and will result inactivation of the relief valve and release of fluid until internal cuff pressure decreases to a point that the valve automatically closes.

13. The catheter system of claim 8, wherein the material of which the fixed-volume inflatable retention cuff (20) is formed is selected from group consisting of nylon, PVC, polyethylene, PET, silicone with nylon fiber reinforcement, silicone with nylon web reinforcement, silicone with polyester fiber reinforcement, silicone with polyester web reinforcement, polyurethane with polyester fiber reinforcement, polyurethane with polyester web reinforcement and polyurethane with nylon reinforcement.

14. The catheter system of claim 9, wherein the relief valve (23, 123) is a one-way valve.
